(19) United States
(12) Patent Application Publication
(10) Pub. No.: US 2010/0168191 A1
(43) Pub. Date: Jul. 1, 2010

(54) PHARMACEUTICAL FORMULATION COMPRISING PRAMIPEXOLE

(75) Inventors: Juergen Reess, Ulm (DE); Thomas Friedl, Ochsenhausen (DE); Nantharat Pearnchob, Biberach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/601,403

(22) PCT Filed: May 10, 2008
(86) PCT No.: PCT/EP2008/003799

§ 371 Date: Dec. 18, 2009

Related U.S. Application Data
(60) Provisional application No. 60/940,134, filed on May 25, 2007.
(60) Provisional application No. 60/957,517, filed on Aug. 23, 2007.

(51) Int. Cl. A61K 31/428; A61P 25/00

(52) U.S. Cl. 514/367

(57) ABSTRACT

The present invention refers to the use of a medicament for the paediatric treatment of RLS and/or Tic Disorder and/or Tourette's Syndrom.

The present invention refers to the use of a medicament for the paediatric treatment of RLS and/or the syndrome complex called Tic Disorder, in particular Tourette's Syndrom.

### BACKGROUND

Idiopathic Restless Leg Syndrome, also known as RLS, anxietas tibiarum, Wittmaack-Ekbom-Syndrom, often called paresthesias (abnormal sensations) or dysesthesias (unpleasant abnormal sensations), is a neurological disorder which manifests itself chiefly as sensory disorders of the legs such as tingling, dragging, tearing, itching, burning, cramp or pain and in those affected triggers an irresistible compulsion to move. These sensations usually occur deep inside the leg, between the knee and ankle; more rarely, they occur in the feet, thighs, arms, and hands. Although the sensations can occur on just one side of the body, they most often affect both sides.

Frequently these sensations occur when the affected person is resting. Particularly at night, during sleep, these sensations and the subsequent compulsive movements lead to restlessness and sleep disturbances. As a result, most people with RLS have difficulty falling asleep and staying asleep. Left untreated, the condition causes exhaustion and daytime fatigue. Many people with RLS report that their job, personal relations, and activities of daily living are strongly affected as a result of their exhaustion. They are often unable to concentrate, have impaired memory, or fail to accomplish daily tasks.

The symptoms of RLS vary in severity and duration from person to person. Mild RLS occurs episodically, with only mild disruption of sleep onset, and causes little distress. In moderately severe cases, symptoms occur only once or twice a week but result in significant delay of sleep onset, with some disruption of daytime function. In severe cases of RLS, the symptoms occur more than twice a week and result in burdensome interruption of sleep and impairment of daytime function.

The disease may begin at any time in life. Usually, the disease is a chronic disease, which starts in a mild form, but usually the symptoms severity increases over time.

The disease may be associated with or patients may develop further conditions, f.e. patients also may suffer from periodic limb movement disorder (PLMD). PLMD is characterized by involuntary leg twitching or jerking movements during sleep that typically occur every 10 to 60 seconds, sometimes throughout the night. The symptoms cause repeated awakening and severely disrupted sleep. Unlike RLS, the movements caused by PLMD are involuntary, meaning the patient has no control over them. Although many patients with RLS also develop PLMD, most people with PLMD do not experience RLS.

Tic Disorders: A tic is an abrupt repetitive movement, gesture, or utterance that often mimics a normal type of behaviour. Motor tics include movements such as eye blinking, head jerks or shoulder shrugs, but can vary to more complex purposive-appearing behaviours such as facial expressions of emotion or meaningful gestures of the arms and head. In extreme cases, the movement can be obscene (copropraxia) or self-injurious. Phonic or vocal tics range from throat clearing sounds to complex vocalizations and speech, sometimes with coprolalia (obscene speech). Tics are irregular in time, though consistent regarding the muscle groups involved. Characteristically, they can be suppressed for a short time by voluntary effort. For an extended definition of tics, and therewith Tic Disorder, it is referred to the DIAGNOSTIC AND STATISTICAL MANUAL OF MENTAL DISORDERS, 4^{th} edition (DSM-IV-TR) of the American Psychiatric Association, pages 108 to 111 and page 114, section 307.22 to page 116 section 307.20, all of which herewith are incorporated by reference.

Gilles de La Tourette syndrome (Tourette's or TS) is an inherited neuro-psychiatric disorder with onset in childhood, characterized by the presence of multiple physical (motor) tics and at least one vocal (phonic) tic; these tics characteristically wax and wane. Tourette's is defined as part of a spectrum of Tic Disorders, which includes transient and chronic tics. For an extended definitions of tics it is referred to the DIAGNOSTIC AND STATISTICAL MANUAL OF MENTAL DISORDERS, 4^{th} edition (DSM-IV-TR) of the American Psychiatric Association, pages 111 to 114, all of which herewith are incorporated by reference. Tourette's syndrome is 3-4 times more common in boys than girls and 10 times more common in children and adolescents than in adults. Among the early symptoms of this conditions are motor tics, for example, eye blinking or head jerks. Initially, tics may come and go, but in time tics become persistent and severe, and begin to have adverse effects on the child and the child's family. Phonic tics manifest, on average, 1 to 2 years after the onset of motor tics. By the age of 10, most affected children have developed an awareness of the premonitory urges that frequently precede a tic. Such premonitions may enable the individual to voluntary suppress the tic, yet premonition unfortunately adds to the discomfort associated with having the disorder. By late adolescence/early adulthood, tic disorders can improve significantly in certain individuals. However, adults who continue to suffer from tics often have particularly severe and debilitating symptoms.

Tic Disorder is estimated to affect 1% to 13% of boys and 1% to 11% of girls, the male-female ratio being less than 2 to 1. Approximately 5% of children between the ages of 7 and 11 years are affected with tic behaviour. The estimated prevalence of multiple tics with vocalization, e.g., Tourette's syndrome, varies among different reports, ranging from 5 per 10,000 to 5 per 1,000.

A medicament with Pramipexole dihydrochloride, preferably pramipexole dihydrochloride monohydrate is known in the US under the tradename MIRAPEX® and in Europe under the tradenames Mirapexin® and Sifrol®. The drug is available in form of tablets that contain pramipexole, a dopamine agonist indicated for the treatment of the signs and symptoms of idiopathic Parkinson's Disease and RLS. The chemical name of pramipexole dihydrochloride is (S)-2-amino-4,5,6,7-tetrahydro-6-(propylamino)benzothiazole dihydrochloride monohydrate. Its empirical formula is C10 H17 N3 S.2HCl.H2O, and its molecular weight is 302.27. The structural formula of the free base is:

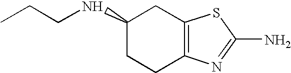

Pramipexole is a nonergot dopamine agonist with high relative in vitro specificity and full intrinsic activity at the D2 subfamily of dopamine receptors, binding with higher affinity to D3 than to D2 or D4 receptor subtypes. The relevance of D3 receptor binding in Parkinson's disease is unknown. If not defined otherwise, in the context of this description and for the claims, the term pramipexole shall include the currently used active ingredient (S)-2-amino-4,5,6,7-tetrahydro-6-(propylamino)benzothiazole dihydrochloride monohydrate as well as any other bioequivalent forms of the drug substance, in particular any pharmaceutically acceptable salt or solvate form other than (S)-2-amino-4,5,6,7-tetrahydro-6-(propylamino)benzothiazole dihydrochloride monohydrate.

### SUMMARY OF THE INVENTION

It is one objective of the present invention to provide a medicament for the treatment of RLS in children.

It is another objective of the present invention to provide a medicament for the treatment of Tic Disorder, here especially in view of motor and vocal tics, in children.

It is another objective of the present invention to provide a medicament for the treatment of Gilles de La Tourette Syndrome in children.

### DETAILED DESCRIPTION

As mentioned above, the term “pramipexole” as used in the context of this description and for the claims refers to (S)-2-amino-4,5,6,7-tetrahydro-6-(propylamino)benzothiazole and pharmaceutically acceptable salts thereof in particular the dihydrochloride monohydrate thereof, if not defined otherwise.

The term “children” refers to children, preferably in the age of 6 years to 18 years, more preferably in the age from 6 years to 17 years. Also preferred are patient collectives in the range of age selected from 6 years to 16 years or 6 years to 15 years or 6 years to 14 years or 6 years to 13 years or 6 years to 12 years.

The invention preferably is carried out with a formulation comprising pramipexole in a dosage suited for oral intake by children as defined above. The dosage of the active ingredient pramipexole is adopted to the needs and pharmacological profile of the active ingredient in children.

In the pharmaceutical formulation, pramipexole may be available in an amount, that allows to apply the recommended daily dosage (see below). Preferred are formulations comprising pramipexole in an amount that corresponds to 0.06 mg to 0.09 mg, and/or 0.03 mg to 0.05 mg, and/or 0.01 mg to 0.029 mg of pramipexole free base.

A preferred formulation contains 0.125 mg, 0.0625 mg or 0.03125 mg of pramipexole dihydrochloride monohydrate as active ingredient, corresponding to 0.088 mg, 0.044 mg, 0.022 mg of the free base. The preferred formulation is a tablet.

Beside (S)-2-amino-4,5,6,7-tetrahydro-6-(propylamino)benzothiazole dihydrochloride monohydrate, other bioequivalent forms of the drug substance may be taken as well. As excipients preferably mannitol, corn starch, colloidal silicon dioxide, povidone, and magnesium stearate are being used. If the term corn starch is used, it may be dried and/or undried.

Mannitol is used as filling agent, corn starch is used as binder and disintegrant, povidone is used as binder, colloidal silicon dioxide is used as glidant and magnesium stearate as lubricant.

The tablet is to be taken 1 to 3 times daily depending on the indication and the age of the children. For RLS, a once daily application is preferred, preferably prior to bedtime. The preferred daily dosage is between 0.01 and 0.5 mg, preferably 0.1 and 0.3 mg, in view of the tablet strength outlined above it is 0.125 mg or 0.25 mg.

For Tourette's as well as for Tic Disorder, a once, a twice or thrice daily application is recommended, preferably a thrice daily evenly distributed over the day (waking hours). The preferred daily dosage is between 0.01 and 0.75 mg. However a dosage between 0.01 and 0.5 mg is preferred, also preferred are dose ranges between 0.1 and 0.4 mg. In view of the tablet strength outlined above three times 0.125 mg or three times 0.0625 mg is preferred.

Tablets may be packaged in aluminium-aluminium blisters or plastic bottles (preferably HDPE, the inner surface of which is darkened, preferably blacked by the addition of suitable additives). In one embodiment the package comprising the tablets may comprise a leaflet in which the recommended daily dose is mentioned.

In another embodiment the package comprising the tablets may comprise a leaflet in which the indication is listed.

In yet another embodiment the package comprising the tablets may comprise a leaflet in which children are mentioned as the recipient for the therapy.

In yet another embodiment the package comprising the tablets may comprise a leaflet in which the daily dosage and/or the indication(s) and/or children as recipient of the therapy is (are) mentioned.

In the following the formulation which preferably can be taken in connection with the present invention is exemplified, while not meant to be limiting, with respect to ingredients or the exact amount of active ingredient.

| | | |
|---|---|---|
| | Composition | |
| Component | [%] | [mg/tablet] |

Pramipexole
0.125 (0.088) mg

| | | |
|---|---|---|
| Pramipexole dihydrochloride | 0.147 | 0.125 |
| monohydrate | | |
| Mannitol | 58.182 | 49.455 |
| Corn Starch, dried | 29.424 | 25.010 |
| Povidone (K 25) | 1.106 | 0.940 |
| Corn Starch | 8.588 | 7.300 |
| Colloidal Silicon Dioxide | 1.106 | 0.940 |
| Magnesium Stearate | 1.447 | 1.230 |
| Sum | 100 | 85.000 |

Pramipexole
0.0625 (0.044) mg

| | | |
|---|---|---|
| Pramipexole dihydrochloride | 0.1042 | 0.0625 |
| monohydrate | | |
| Mannitol | 57.9417 | 34.7650 |
| Corn Starch, dried | 29.4120 | 17.6472 |
| Povidone (K 25) | 1.1917 | 0.7150 |
| Corn Starch | 8.5880 | 5.1528 |
| Colloidal Silicon Dioxide | 1.1917 | 0.7150 |
| Magnesium Stearate | 1.5708 | 0.9425 |
| Sum | 100 | 60.000 |

Pramipexole
0.03125 (0.022) mg

| | | |
|---|---|---|
| Pramipexole dihydrochloride | 0.0521 | 0.03125 |
| monohydrate | | |
| Mannitol | 57.9938 | 34.79625 |
| Corn Starch, dried | 29.4120 | 17.6472 |
| Povidone (K 25) | 1.1917 | 0.7150 |
| Corn Starch | 8.5880 | 5.1528 |
| Colloidal Silicon Dioxide | 1.1917 | 0.7150 |
| Magnesium Stearate | 1.5708 | 0.9425 |
| Sum | 100 | 60.000 |

1. A method for treating RLS in a child, comprising administering to a child suffering from RLS a per day dosage of pramipexole dihydrochloride monohydrate between 0.01 mg and 0.75 mg.

2. A method according to claim 1, wherein the per day dosage of pramipexole dihydrochloride monohydrate is between 0.01 mg and 0.5 mg.

3. (canceled)

4. (canceled)

5. (canceled)

6. (canceled)

7. A method according to claim 1, wherein the pramipexole dihydrochloride monohydrate as active ingredient is contained in a pharmaceutical formulation further comprising mannitol, corn starch, colloidal silicon dioxide, povidone, and magnesium stearate.

8. A method according to claim 7, wherein the pharmaceutical formulation is a tablet comprising pramipexole dihydrochloride monohydrate as active ingredient, mannitol, corn starch, colloidal silicon dioxide, povidone, and magnesium stearate, which tablet is contained in a package for the tablet and the package further includes a leaflet indicating that the intended patient group are children.

9. A method for treating RLS in a child, comprising administering to a child suffering from RLS a pharmaceutical formulation comprising pramipexole free base or a pramipexole salt form in an amount that corresponds to 0.06 mg to 0.09 mg of (S)-2-amino-4,5,6,7-tetrahydro-6-(propylamino)benzothiazole.

10. (canceled)

11. (canceled)

12. (canceled)

13. A method according to claim 9, wherein the pramipexole free base or pramipexole salt form is present in an amount that corresponds to 0.088 mg of (S)-2-amino-4,5,6,7-tetrahydro-6-(propylamino)benzothiazole.

14. A method for treating RLS in a child, comprising administering to a child suffering from RLS a pharmaceutical formulation comprising pramipexole free base or a pramipexole salt form in an amount that corresponds to 0.03 mg to 0.05 mg of (S)-2-amino-4,5,6,7-tetrahydro-6-(propylamino)benzothiazole.

15. A method according to claim 14, wherein the pramipexole free base or pramipexole salt form is present in an amount that corresponds to 0.044 mg of (S)-2-amino-4,5,6,7-tetrahydro-6-(propylamino)benzothiazole.

16. A method for treating RLS in a child, comprising administering to a child suffering from RLS a pharmaceutical formulation comprising pramipexole free base or a pramipexole salt form in an amount that corresponds to 0.01 mg to 0.029 mg of (S)-2-amino-4,5,6,7-tetrahydro-6-(propylamino)benzothiazole.

17. A method according to claim 16, wherein the pramipexole free base or pramipexole salt form is present in an amount that corresponds to 0.022 mg of (S)-2-amino-4,5,6,7-tetrahydro-6-(propylamino)benzothiazole.
